(19) United States
(12) Patent Application Publication
(10) Pub. No.: US 2006/0217600 A1
(43) Pub. Date: Sep. 28, 2006

(54) Refrigerator for health management

(76) Inventors: Ik Kyu Lee, Gimhae-si (KR); Jae Wook Jun, Jinju-si (KR); Hee Soo Son, Busan (KR); Jae Hoon Jung, Gimhae-si (KR)

Correspondence Address: FLESHNER & KIM, LLP, P.O. BOX 221200, CHANTILLY, VA, 20153, US

(21) Appl. No.: 11/384,810

(22) Filed: Mar. 21, 2006

(30) Foreign Application Priority Data
Mar. 22, 2005 (KR) P2005-0023726
Jul. 6, 2005 (KR) P2005-0060651
Jul. 29, 2005 (KR) P2005-0069823
Sep. 8, 2005 (KR) P2005-0083887

(51) Int. Cl. A61B 5/00; F25D 23/12; A61B 5/04; F25D 15/00

(52) U.S. Cl. 600/300; 600/372; 62/331

(57) ABSTRACT

A refrigerator for health management is disclosed, by which user's health is efficiently managed to prevent corpulence and other diseases of adult people. The present invention includes an input device for inputting user data, a controller computing user's health information based on the user data, and a display device for displaying the health information to the user.

This application claims the benefit of the Korean Patent Application No. P2005-0023726 filed on Mar. 22, 2005, No. P2005-0060651 filed on Jul. 6, 2005, No. P2005-0069823 filed on Jul. 29, 2005, and No. P2005-0083887 filed on Sep. 8, 2005, which are hereby incorporated by reference as if fully set forth herein.

### BACKGROUND OF THE INVENTION

1. Field of the Invention

The present invention relates to a refrigerator, and more particularly, to a refrigerator for health management. Although the present invention is suitable for a wide scope of applications, it is particularly suitable for managing user's health efficiently.

2. Discussion of the Related Art

Generally, a refrigerator is a device that keeps food fresh in a low temperature state for a long term by maintaining a freezer room and a cold storage room at low temperatures using chilly drafts, respectively. And, refrigerators are globally provided according to massive demand and supply due to the development of the industrial society.

Meanwhile, most of the diseases taking place in the modern industrial society mostly rise from wrong eating habits, let alone such an indirect influence as environmental pollution and the like. Particularly, diseases of adult people rising from corpulence are rapidly increasing nowadays.

Meanwhile, an abdominal fat ratio is raised by the excessive fat (internal organ fat) surrounding internal organs, whereby a risk of such a disease as high blood pressure, cardiovascular disease and the like is considerably raised.

So, the demand for a function of managing user's health totally in a manner of preventing the corpulence by managing user's weight via a refrigerator keeping food in a low temperature state rises.

### SUMMARY OF THE INVENTION

Accordingly, the present invention is directed to a refrigerator that substantially obviates one or more problems due to limitations and disadvantages of the related art.

An object of the present invention is to provide a refrigerator, by which user's health is efficiently managed.

Additional advantages, objects, and features of the invention will be set forth in part in the description which follows and in part will become apparent to those having ordinary skill in the art upon examination of the following or may be learned from practice of the invention. The objectives and other advantages of the invention may be realized and attained by the structure particularly pointed out in the written description and claims hereof as well as the appended drawings.

To achieve these objects and other advantages and in accordance with the purpose of the invention, as embodied and broadly described herein, a refrigerator for health management according to the present invention includes an input device for inputting user data, a controller computing user's health information based on the user data, and a display device for displaying the health information to the user.

Preferably, the user data inputted by the input device includes at least one of a height of the user, a weight of the user, a sex of the user, user recognizing information and body composition information.

Preferably, the input device includes at least one of a data input unit inputting the user data by the user, a scale detachably provided to the refrigerator, a sensor unit recognizing the user and a measuring unit measuring body components.

More preferably, the controller includes a data processing unit computing health information based on the user data inputted by the input device and a data storing unit accumulating and storing at least one of the user data inputted by the input device and the health information computed by the data processing unit.

More preferably, the data input unit includes a mode switching unit for entering a health management mode.

More preferably, the scale is loaded in an accommodating part provided to a body of the refrigerator.

More preferably, the scale includes an input terminal transferring information measured by being selectively contacted with a connection terminal provided to one side to the controller.

More preferably, the scale includes a connecting wire connected to the controller.

More preferably, the connecting wire passes through a hinge portion of a door of the refrigerator.

More preferably, the sensor unit comprising at least one of a fingerprint recognizing sensor for inputting fingerprint information of the user and an iris recognizing sensor for inputting iris information of the user.

More preferably, the controller decides whether a preset value passes since a latest time of measuring the user recognized by the sensor unit through the scale or the measuring unit. If the preset value passes, the controller transfers an announcement signal inducing a measurement of the user to the display device.

Preferably, the display device displays a character according to the health information.

More preferably, the measuring unit measures body components of the user based on an electric resistance of a human body.

More preferably, the measuring unit is provided to a door handle of the refrigerator.

More preferably, the measuring unit includes a contact member configured to be outwardly projected, the contact member formed of an electric conductor coming into contact with a body of the user, an elastic member elastically supporting the contact member, and a connecting terminal selectively coming into contact with the contact member to guide a current to the contact member.

Preferably, the user's health information computed by the controller includes at least one of a menu corresponding to the health information, information for a corpulence degree and information for an exercise method.

It is to be understood that both the foregoing general description and the following detailed description of the present invention are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this application, illustrate embodiment(s) of the invention and together with the description serve to explain the principle of the invention. In the drawings:

FIG. 1 is a perspective diagram of a refrigerator according to a first embodiment of the present invention;

FIG. 2 is a magnified perspective diagram of a connecting mechanism of a scale shown in FIG. 1;

FIG. 3 is a block diagram of a refrigerator according to a first embodiment of the present invention;

FIG. 4 is a magnified perspective diagram of a modified connecting mechanism of a scale shown in FIG. 1;

FIG. 5 is a flowchart of a method of controlling a refrigerator according to a first embodiment of the present invention;

FIG. 6 is a flowchart of another method of controlling a refrigerator according to a first embodiment of the present invention;

FIG. 7 is a perspective diagram of a refrigerator according to a second embodiment of the present invention;

FIG. 8 is a cross-sectional diagram of a body fat measuring sensor shown in FIG. 7;

FIG. 9 is a block diagram of a refrigerator according to a second embodiment of the present invention; and

FIG. 10 is a flowchart of a method of controlling a refrigerator according to a second embodiment of the present invention.

### DETAILED DESCRIPTION OF THE INVENTION

Reference will now be made in detail to the preferred embodiments of the present invention, examples of which are illustrated in the accompanying drawings. Wherever possible, the same reference numbers will be used throughout the drawings to refer to the same or like parts.

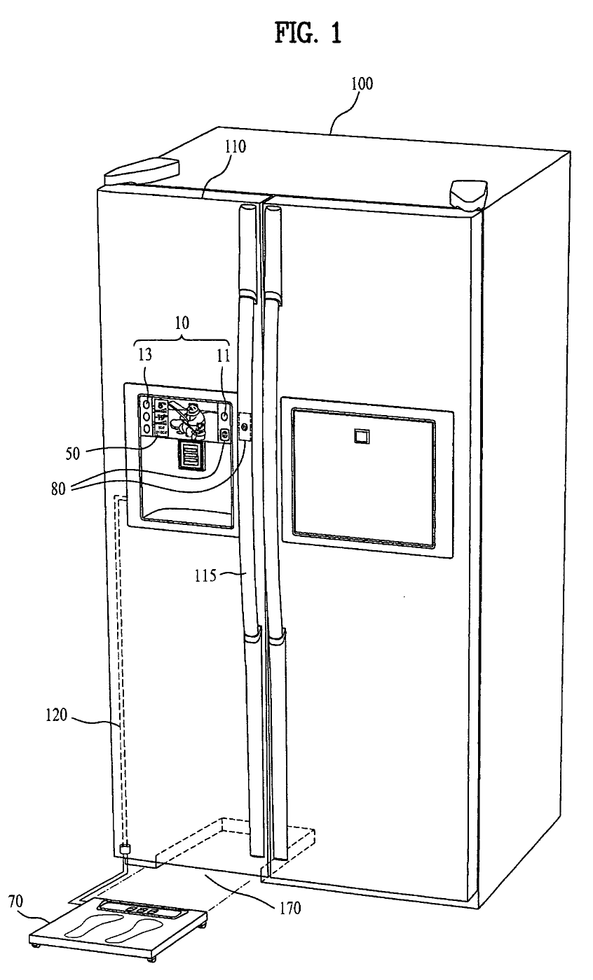

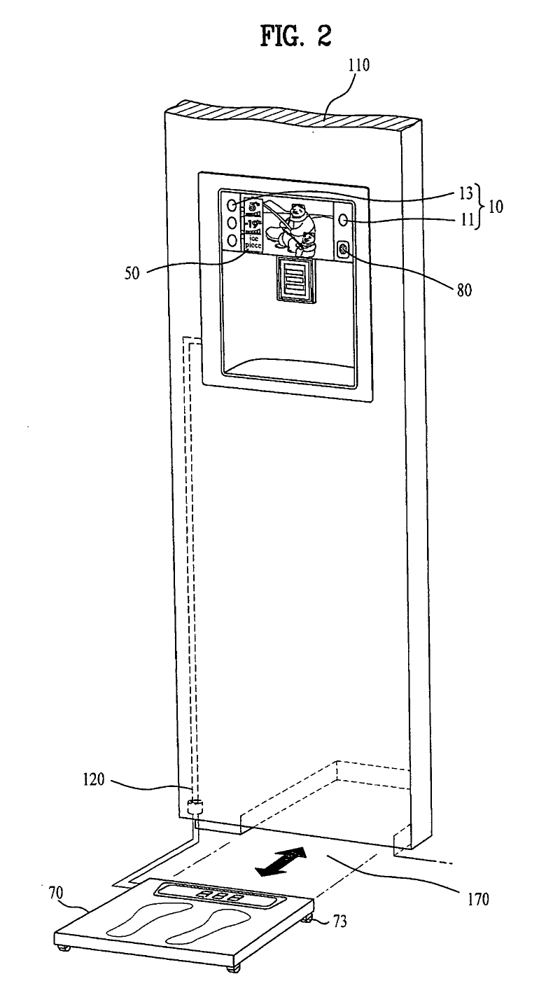

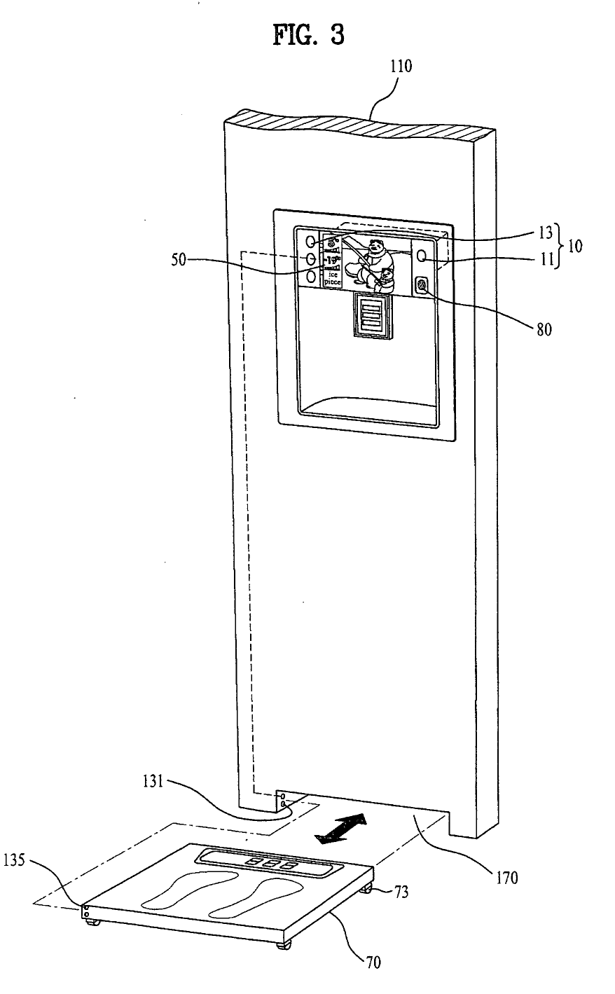

First of all, a configuration of a refrigerator for health management according to a first embodiment of the present invention is explained with reference to FIGS. 1 to 3 as follows.

In a general refrigerator, a pair of doors 110 are rotatably assembled to both sides of a refrigerator body 100 having a freezer room and a cold storage room. A controller 30 is provided within the refrigerator body 100 to control a chilly draft to be generated and supplied to the freezer room and the cold storage room. And, an input device 10 adjusting an operational state of the refrigerator and a display device 50 displaying the operational state of the refrigerator are installed on the refrigerator door 110.

Meanwhile, a dispenser facilitating ice or cold water to be supplied to a user is provided to the refrigerator door 110.

For user's health management, the refrigerator according to an embodiment of the present invention includes an input device 10 inputs user data, a controller 30 computing health information useful for user's health management based on the user data inputted via the input device 10, and a display device 50 displaying the user's health information computed by the controller 30.

Te input device 10 includes a mode switching unit 11 entering a mode for weight management and a data input unit 13 inputting various user data in a health management mode entered by the mode switching unit 11.

In this case, the mode switching unit 11 switches a temperature management mode for adjusting temperatures of a freezer room and a cold storage room to the health management mode displaying a user's health state according to the user data and is configured to enable entries into various modes as well as the temperature management mode and the health management mode.

Even if the health management mode is entered by being selected by the mode switching unit 11, the temperatures of the freezer and cold storage rooms keep being controlled according to the values set by the temperature management mode.

The data input unit 13 is configured to input various user data such as a user's height, a user's weight and the like in the health management mode. Yet, to compute more accurate health information, the user data can include a chest girth, a lower chest girth, a belly girth, a hip girth, a thigh girth, a nipple interval, a breast position, a shoulder width, a hip height and the like. Preferably, the user data further includes a sex as well as the above-illustrated user data to enable the health information to be more accurately computed.

The mode switching unit 11 and the data input unit 13 used in the health management mode preferably share various buttons used for the user to switch the operational state of the refrigerator from the temperature management mode arbitrarily. Yet, they can use different buttons, respectively.

The controller 30 is a device computing the health information useful for a user using the various user data inputted by the input device 10. In particular, the controller 30 performs a function of computing a corpulence rate according to the inputted user data and a function of selecting an optimal menu corresponding to the user data.

The controller 30 includes a data processing unit 33 computing the health information according to the user data inputted by the data input unit 13 and a data storing unit 31 accumulating to store the user data inputted by the input device 10 and at least one of the health information computed by the data processing unit 33.

The data processing unit 33 stores a corpulence rate computing formula for computing the corpulence rate or a table about the corpulence rate according to the data such as the height, the weight, the sex and the like in advance. Hence, a user's corpulence rate can be computed from the corpulence rate computing formula or the table about the corpulence rate.

The data storing unit 31 is configured to accumulate and store the health information such as the user data and the corpulence rate by considering a measuring time according to a user. By accumulating to store various data per user, a reference for managing user's health can be provided.

Besides, various healthful menus, recipes for the menus and the like are stored in the data storing unit 31 to enable the data processing unit 33 to select an optimal healthful menu created to include appropriate nutritive elements by reducing body fat and sugar based on the health information computed to correspond to the user data by the data processing unit.

The display device 50 plays a role in displaying the user's health information computed by the controller 30 to the user and generally includes a liquid crystal display (LCD) device 50.

The display device 50 can represent a corpulence rate as a numerical value or various type characters. The display device 50 can represent the corpulence rate and body shape variation as a graph or the like. And, the display device can represent information for an optimal healthful menu corresponding to a user's corpulence rate, a corresponding recipe, a work-out program and the like as characters or various pictures.

The character information represents a user's weight as a figure in a manner of differentiating an area of a human body figure or displays an increment/decrement of the user's weight in a manner of expanding or reducing a body part of an animal character set by each user.

Preferably, the refrigerator according to the present invention further includes a scale 70 measuring a user's weight to provide information of the measured weight to the controller 30.

As mentioned in the foregoing description, a user can directly input her/his weight to the input device 10. To measure the user's weight more precisely and to keep maintaining a variation of each user's weight continuously, the refrigerator according to the embodiment of the present invention includes the scale 70 separately.

The scale 70 is configured to measure a weight pressurized by a user using a spring, a strain gauge or the like.

And, the scale 70 of the refrigerator according to the embodiment of the present invention is detachably provided to an accommodating part 170 provided to a lower end of the refrigerator.

A front side of the accommodating part 170 is open to enable the scale 70 be inserted therein and is configured to be recesses inward. Since a plurality of legs are provided to a refrigerator in general to support a bottom surface of the refrigerator in a manner of spacing the bottom surface apart from a ground or the like, the accommodating part 170 is preferably provided to a space between the ground and the bottom surface of the refrigerator.

Meanwhile, in the present embodiment, the controller 30 and the scale 70 are connected together by a connecting wire 120 passing through a hinge portion of the refrigerator door 110.

The connecting wire 120 supplies a power to the scale 70 including the strain gauge and provides user's weight information measured by the scale 70 to the controller 30.

In the present embodiment, the connecting wire 120 mutually connects the power of the refrigerator, the controller 30 and the scale 70 loaded in the lower end of the refrigerator and is configured to pass through an opening of a hinge center of the refrigerator door 110.

In this case, the connecting wire 120 is formed longer than a length in case of loading the scale 70 in the refrigerator to maintain a connected state between the scale 70 and the refrigerator even if the scale 70 is drawn out of the accommodating part.

The scale 70 is configured to have a plate shape on which a user can stand 70. And, the scale 70 is provided with a plurality of wheels 73 to be movable.

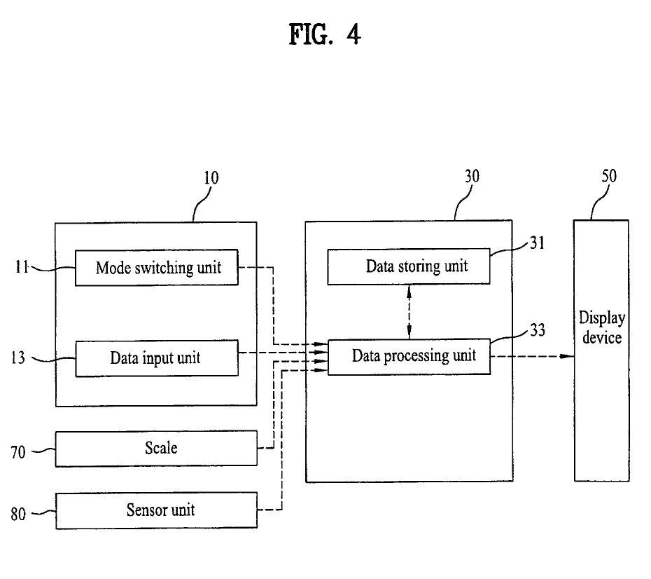

FIG. 4 is a magnified perspective diagram of a modified connecting mechanism of a scale shown in FIG. 1.

Referring to FIG. 4, the controller 30 and the scale 70 are selectively connected to each other via a pair of connection and input terminals 135 and 131.

IN particular, to provide user's weight information measured by the scale 70 to the controller 30, the connection terminal 135 is provided to one side of the scale 70 and the input terminal 131 is provided to a portion of the accommodating part 170 corresponding to the connection terminal 135 to enable a selective contact with the connection terminal 135.

Through the above-explained configuration, the input terminal 131 is connected to a circuit provided within the scale 70 by the connection terminal 135 to transfer the user's weight information measured by the scale 70 to the controller 30.

Alternatively, the controller 30 and the scale 70 are connected to each other by a wireless communication device such as a local wireless communication device, an RF communication device and the like to transfer the user's weight information measured by the scale 70 to the controller 30 by wireless.

In the present embodiment, the refrigerator includes a sensor unit 80 sensing a user by receiving information for the user.

The sensor unit 80 can be configured in various types. In the present invention, the sensor unit 80 includes a fingerprint recognizing sensor that inputs user's fingerprint information. Alternatively, the sensor unit 80 can include an iris recognizing sensor inputting user's iris information.

In the above description, the health management mode is entered by the mode selecting unit. Yet, the sensor unit 80 is provided to a front side of the refrigerator door 110. So, in case that a user grabs or manipulates a door handle 115 to use the refrigerator, the sensor unit 80 provided to the door handle 115 grabbed by the user recognizes the user to enter the health management mode.

Meanwhile, the controller 30 decides whether a preset time expires from a latest time of measuring a weight of a user recognized by the sensor unit 80.

As a result of the decision, if the preset time expires, an announcement signal inducing a user's weight measurement is transferred to the display device 50.

The display device 50 displays a character display or sound guiding a user to measure her/his weight according to the announcement signal.

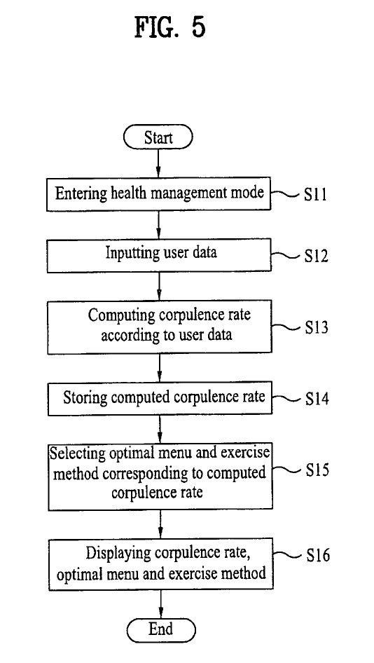

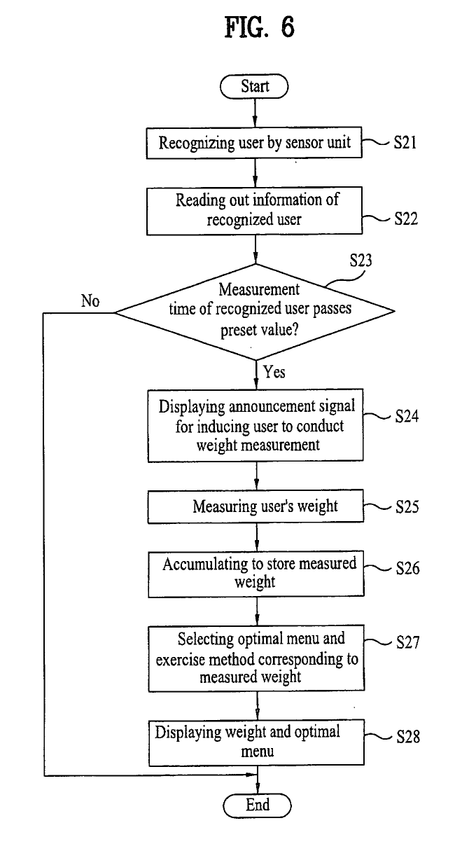

A method of controlling the above-configured refrigerator according to the embodiment of the present invention is explained with reference to FIG. 5 and FIG. 6 as follows.

First of all, a method of controlling a refrigerator in managing a user's weight using user data inputted by the user only instead of using the scale 70 and the sensor unit 80 is explained with reference to FIG. 5 as follows.

Referring to FIG. 5, a user enters a health management mode for managing a user's weight by the mode switching unit 11 (S11). And, various user data including user's height, weight, sex and the like are inputted by the data input unit 13 in a health management mode (S12).

In doing so, the user data inputted in the health management mode are preferably accumulated and stored in the data storing unit 31.

In particular, a signal is generated each time the mode switching unit 11 or the data input unit 13 is pressed and is then transferred to the controller 30. Hence, various user data are inputted in the health management mode.

Subsequently, a users corpulence rate is computed by the data processing unit using the user data inputted by the data input unit 13 (S13). The computed corpulence rate is then stored in the data storing unit 31 (S14).

In doing so, various body sizes are accumulated and stored per each user in the data storing unit 31. Simultaneously, the corresponding corpulence rate is preferably accumulated and stored in the data storing unit 31. And, the stored health information becomes an important reference for user's health management.

An optimal menu and exercise method are selected from various preset menus to correspond to the corpulence rate computed according to the user data (S15). And, the corpulence rate, optimal menu and exercise method are displayed on the display unit 50 (S16).

In this case, it is preferable that at least two menus are selected as the optimal menus to offer an opportunity of selecting various menus to user.

A method of controlling a refrigerator for managing a user's weight using the scale 70 and the sensor unit 80 provided to the refrigerator is explained with reference to FIG. 6 as follows.

Referring to FIG. 6, a user is recognized by the sensor unit 80 to initiate a health management mode (S21). User information recognized by the sensor unit 80 is transferred to the controller 30 to read out weight information corresponding to the recognized user from the data storing unit 31 of the controller 30 (S22).

Through weight measurement time included in the recognized user information, the controller 30 decides whether a measurement time of the recognized user exceeds a preset value by comparison (S23). As a result of the decision, if the measurement time of the user exceeds the preset value, an announcement signal inducing the user to measure her/his weight to the display device 50. The display device 50 then displays a character display or sound inducing the user to conduct a weight measurement externally (S24).

Subsequently, the user having recognized the announcement signal of the display device 50 draws the scale 70 out of the accommodating part 170 of the refrigerator and then measures the weight (S25). The measured user's weight information is accumulated and stored in the data storing unit 31 per user to enable user's weights to be continuously managed (S26).

An optimal menu and exercise method are selected from various preset menus and exercise methods to correspond to the measured user's weight (S27) and the measured weight, optimal menu and exercise method are displayed on the display device 50 (S28).

In this case, the user's weight information is displayed by a time unit to be recognized by being compared to previously stored user's weight information or is represented as a character or the like to facilitate the user to recognize the information. Thus, it is preferable that the user can easily recognize the weight information and the corresponding variations.

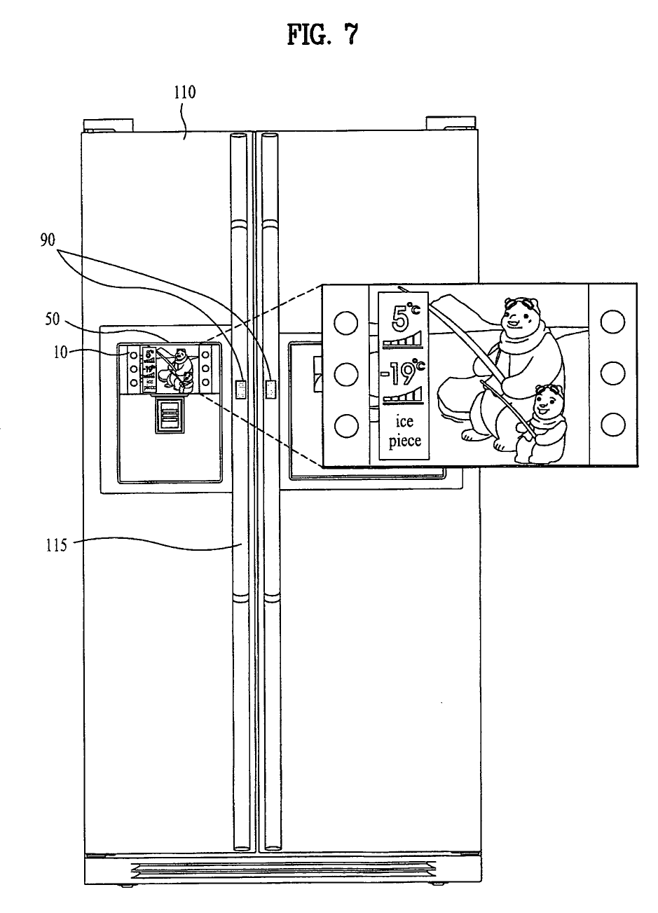

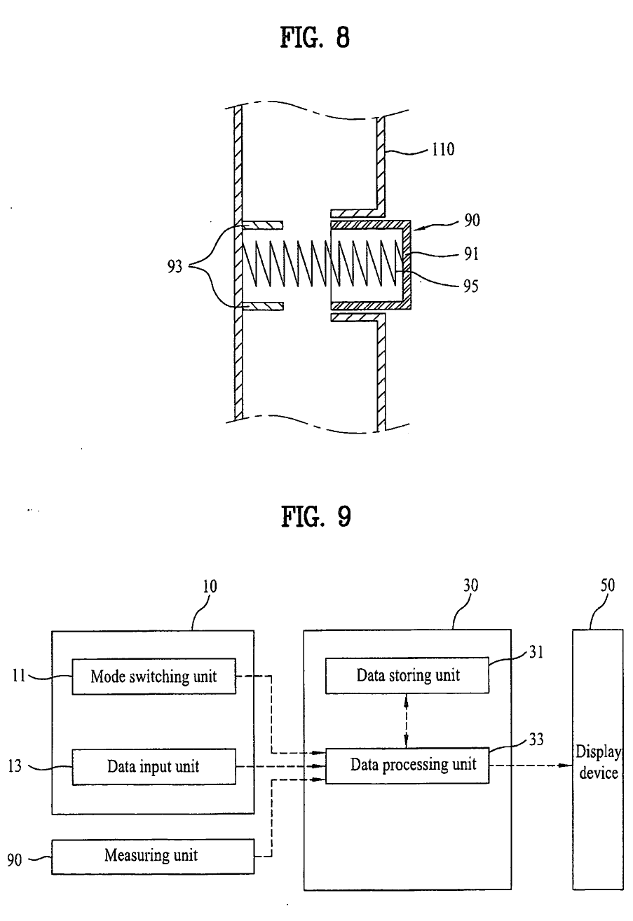

A refrigerator according to a second embodiment of the present invention is explained with reference to FIGS. 7 to 9 as follows.

First of all, a refrigerator according to a second embodiment of the present invention includes an input device 10, a controller 30 and a display device 50.

In the present embodiment, the refrigerator includes a measuring unit 90 provided to a refrigerator door 110 to measure body components instead of the scale 70 detachably provided to the refrigerator and the sensor unit 80 in the former embodiment of the present invention.

The measuring unit 90 performs a function of measuring user's body components. For example, the measuring unit 90 can measure a user's body fat quantity based on an electric resistance of a human body.

Preferably, the measuring unit 90 is provided to a refrigerator handle 115 to facilitate a measurement made by a user.

In the present invention, the measuring unit 90 includes a contact member 91, an elastic member 95 and a connecting terminal 93.

The contact member 91 is outwardly projected and is formed on an electrically conductive material coming into contact with a user's body. A backside of the contact member 91 is configured to be caught on an inside of the handle 115.

The elastic member 95 is a member elastically supporting the contact member 91 and may include a spring for example.

The connecting terminal 93 plays a role in guiding a current to the contact member 91 by coming into contact with the contact member. And, the connecting terminal 93 is installed to be built in the refrigerator handle 115 and to leave a predetermined gap from the backside of the contact member 91.

So, only if the contact member 91 is compressed by a prescribed length or more, the backside of the contact member 91 is contacted with the connecting terminal 93. Once a current passes through the connecting terminal 913, the contact member 91 and the human body in turn from the controller 30, the resistance of the human body can be measured.

For instance, after a current is made to flow to the measuring unit 90 provided to a freezer room door 110 contacted with a right hand palm based on the measuring unit 90 provided to a cold storage room door 110 contacted with a left hand palm, an electric resistance of the human body can be measured by measuring a voltage difference between the measuring unit 90 provided to the freezer room door 110 and a measurement sensor provided to the cold storage room door 110.

In this case, a method of measuring a body fat quantity of a human body by the measuring unit 90 employs a bio-impedance measuring method (BIA) among various methods of measuring body fat. In particular, the bio-impedance measuring method (BIA) is carried out in a manner of measuring a resistance of a human body by applying a weak current of about 250 mA to the human body.

In particular, the current flowing through the human body can pass through water having high electric conductivity only among human body elements such as protein, inorganic substance, fat and the like. So, an electric path is widened to reduce the resistance if water is sufficiently provided within the human body. Otherwise, the electric path is narrowed to increase the resistance. And, a volume of water within the human body can be calculated. Furthermore, a muscle quantity within the human body can be computed based on the physiological theory that 73.3% of muscle comprises water. Hence, a body fat quantity can be calculated by subtracting the computed water quantity and the muscle quantity (protein and inorganic substance) from a body weight.

Preferably, the measuring unit 90 is installed to be connected to the controller 30 to compute the body fat quantity using the physiological impedance measuring method. Preferably, a body fat computing program according to the above-explained physiological impedance measuring method is installed at the controller 30.

Meanwhile, the controller 30 includes the data processing unit 33 computing the body fat quantity according to the human body impedance measured by the measuring unit 90 in the health management mode. The data processing unit 33 analyzes a health state by considering the body fat quantity together with user data inputted by the input device 10 and then provides a corresponding management method.

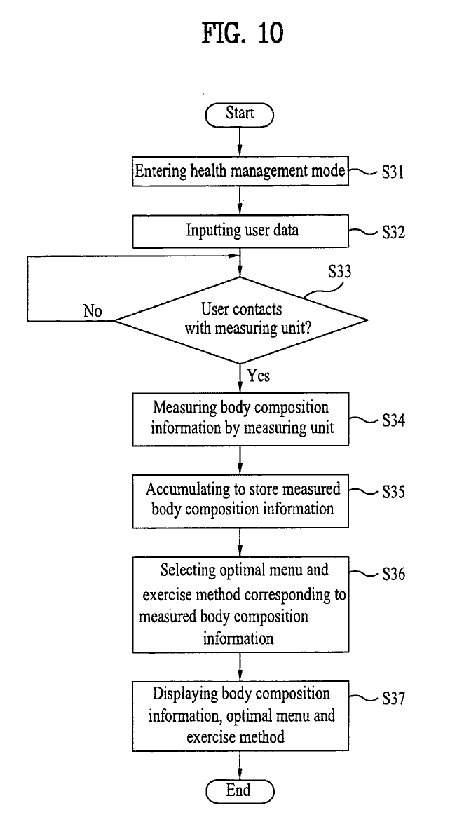

A method of controlling the refrigerator according to the above-explained embodiment of the present invention is explained with reference to FIG. 10 as follows.

Referring to FIG. 10, first of all, a user enters a health management mode for managing a user's weight by the mode switching unit 11 (S31). Various user data including a user's height, weight, sex and the like are inputted by the data input unit 13 in the health management mode (S32).

In this case, the user directly manipulates the mode switching unit 11 to enter the health management mode. Alternatively, it can be configured to automatically enter the health management mode as soon as a human body is contacted with the measuring unit 90.

Subsequently, the measuring unit 90 decides a presence or non-presence of a human body contact (S33). If the human body is contacted with the measuring unit 90, body composition information, i.e., user's body fat quantity is measured through an electric resistance of the human body (S34).

In this case, when the health management mode is entered, a current flows via the measuring unit 90. The presence or non-presence of the human body contact is decided by measuring a resistance according to the current delivered from the measuring unit 90. In particular, it is decided that the human body is contacted only if the electric resistance of the human body measured by the measuring unit 90 is equal to or greater than a prescribed value.

Meanwhile, it is able to acquire a body index such as a per-part body fat and the like as well as a total body fat of human body by configuring various parts of human body to contact with the measuring unit 90 and by applying a virtual human body structure.

The body composition information measured by the measuring unit 90 is accumulated and stored per user in the data storing unit 31 to enable the user's weight to be continuously managed (S35).

The above accumulated and stored data is utilized in diagnosing a corpulence-associated health state in a corpulence management mode later as well as providing a management method, whereby corpulence can be effectively managed for a long term.

An optimal menu is selected from various preset menus to correspond to the user's measured body composition information (S36). The measured body fat quantity and the optimal menu are then displayed on the display device 50 (S37).

Optionally, health information useful for a user can be computed and displayed by considering the inputted user data and the measured body composition information.

The health information useful for the user includes various kinds of information such as information of a user's corpulence degree, a recommended exercise program, menu information and the like.

In particular, the health information useful for the user can include the information for the corpulence degree such as an overall corpulence rate, a per-part corpulence rate, an abdominal corpulence rate and the like. For instance, a body weight index is computed using the height and weight among the user data, the user is classified into a low weight, a normal weight, an overweight and a corpulence according to the body weight index. And, the user can be identified as a partial corpulence by considering the body fat quantity.

And, the health information useful for the user can include information for an exercise management program including a recommended exercise by considering a body fat consumption, a muscle power enhancement and the like according to the inputted user data and the measured body fat quantity, information for motions and effects included in the corresponding recommended exercise, and information for a menu management program including an optimal menu by considering calories and nutritive elements and a food recipe included in the optimal menu.

It is a matter of course that the data processing unit 33 can decide the corpulence-associated health state more specifically by receiving more user data and additional body composition measurement values as well as the body fat quantity.

Accordingly, the present invention provides the following effects or advantages.

First of all, by providing the health information useful for user's weight management according to the user's body data, the user is guided for her/his weight management before eating food from the refrigerator. Hence, the user's weight can be efficiently managed.

Specifically, the user can adjust food ingestion by receiving the information for her/his corpulence rate, a corresponding optimal menu and a corresponding exercise method, whereby corpulence and diseases of adult people can be prevented.

And, the user's health information is represented as figures, characters, sound and the like, thereby enabling the user to recognize the health information with ease.

Secondly, the scale is detachably provided to the refrigerator and various health information is provided to the user according to the weight information measured by the scale. Hence, the weight management can be carried out more efficiently.

Specifically, the accommodation and management of the scale are facilitated and the user is facilitated to recognize her/his weight information. Hence, user's convenience is enhanced.

Thirdly, the user can recognize her/his weight information in using the refrigerator and is induced to measure her/his weight periodically. Hence, the user's weight can be continuously managed.

Fourthly, by providing the measuring unit to an easily accessible and measurable place, the user can acquire her/his body composition information with ease.

Finally, by providing various kinks of health information to the user to correspond to the measured body composition information, the user can manage her/his information more efficiently.

It will be apparent to those skilled in the art that various modifications and variations can be made in the present invention without departing from the spirit or scope of the inventions. Thus, it is intended that the present invention covers the modifications and variations of this invention provided they come within the scope of the appended claims and their equivalents.

1. A refrigerator for health management, comprising:
an input device for inputting user data;
a controller computing user's health information based on the user data; and
a display device for displaying the health information to the user.

2. The refrigerator for the health management of claim 1, wherein the user data inputted by the input device includes at least one of a height of the user, a weight of the user, a sex of the user, user recognizing information and body composition information.

3. The refrigerator for the health management of claim 1, wherein the input device includes at least one of a data input unit inputting the user data by the user, a scale detachably provided to the refrigerator, a sensor unit recognizing the user and a measuring unit measuring body components.

4. The refrigerator for the health management of claim 1, the controller comprising:
a data processing unit computing health information based on the user data inputted by the input device; and
a data storing unit accumulating and storing at least one of the user data inputted by the input device and the health information computed by the data processing unit.

5. The refrigerator for the health management of claim 3, the data input unit comprising a mode switching unit for entering a health management mode.

6. The refrigerator for the health management of claim 3, wherein the scale is loaded in an accommodating part provided to a body of the refrigerator.

7. The refrigerator for the health management of claim 3, the scale comprising an input terminal transferring information measured by being selectively contacted with a connection terminal provided at a body of the refrigerator to the controller.

8. The refrigerator for the health management of claim 3, the scale comprising a connecting wire connected to the controller.

9. The refrigerator for the health management of claim 8, wherein the connecting wire passes through a hinge portion of a door of the refrigerator.

10. The refrigerator for the health management of claim 3, the sensor unit comprising at least of a fingerprint recognizing sensor for inputting fingerprint information of the user and an iris recognizing sensor for inputting iris information of the user.

11. The refrigerator for the health management of claim 10, wherein the controller decides whether a preset value passes since a latest time of measuring the user recognized by the sensor unit through the scale or the measuring unit, and
wherein if the preset value passes, the controller transfers an announcement signal inducing a measurement of the user to the display device.

12. The refrigerator for the health management of claim 1, wherein the display device displays a character according to the health information.

13. The refrigerator for the health information of claim 3, wherein the measuring unit measures body components of the user based on an electric resistance of a human body.

14. The refrigerator for the health management of claim 13, wherein the measuring unit is provided to a door handle of the refrigerator.

15. The refrigerator for the health management of claim 13, the measuring unit comprising:
a contact member configured to be outwardly projected, the contact member formed of an electric conductor coming into contact with a body of the user;
an elastic member elastically supporting the contact member; and
a connecting terminal selectively coming into contact with the contact member to guide a current to the contact member.

16. The refrigerator for the health management of claim 1, wherein the user's health information computed by the controller includes at least one of a menu corresponding to the health information, information for a corpulence degree and information for an exercise method.
